SEQUENCE LISTING (1 sequence)

SEQ ID NO: 1 - DNA, 389 bp, Artificial Sequence
  Description of Artificial Sequence Microsatellite D1S2729 and flanking regions
agctgctgagtttgtagtgatatggttacacagcaatagatgaatatagtgaggaacagtctgtaaagcactgagtccagtgctggcatgtggaggtgctctgtaaggagttgtgttattactgttgtattgtnagtctgctgattacttgcctaatgctgtgtggggcctggctttgccctgccccggtccctagtggggccaggttccatggctctnactagccctgctggttctnataccctggtacagaaagaaagattctatgactcaaacacacacacacacacacacacacacacacacacacacacacacacaccccagagccttaggccttggtctcccaaggattgatatcccagcccagtccacatgattctgaattg

(19) United States
(12) Patent Application Publication
(10) Pub. No.: US 2004/0014056 A1
(43) Pub. Date: Jan. 22, 2004

(54) Identification of genetic markers

(76) Inventors: Jorg Hager, Mennecy (FR); Ivo Glynne Gut, Paris (FR)

Correspondence Address: Nixon & Vanderhye, 8th Floor, 1100 North Glebe Road, Arlington, VA, 22201-4714, US

(21) Appl. No.: 10/258,867

(22) PCT Filed: Apr. 30, 2001
(86) PCT No.: PCT/EP01/04871

(30) Foreign Application Priority Data
May 2, 2000 (EP) 00401202.7

(51) Int. Cl. C12Q 1/68

(52) U.S. Cl. 435/6

(57) ABSTRACT

The present invention relates to a method for the identification of the presence of a genetic marker in a DNA sample, in particular by using a oligonucleotide array. In particular, the method according to the invention allows for the identification and/or localization of gene(s) associated with a distinguishable phenotype.

The present invention relates to a method for the identification of the presence of a genetic marker in a DNA sample, in particular by using a oligonucleotide array. In particular, the method according to the invention allows for the identification and/or localization of gene(s) and/or mutation(s) associated with a distinguishable phenotype.

Definitions

By “complementary”, it is referred to the topological compatibility or matching together of interacting surfaces of a probe molecule and its target Thus, the target and its probe can be described as complementary, and furthermore, the contact surface characteristics are complementary to each other. Although perfect complementarity is preferred, certain mismatch may be tolerated, as long as the specificity of hybridization is retained.

As used herein, “isolated” includes reference to material which is substantially or essentially free from components which normally accompany or interact with it as found in its naturally occurring environment. The isolated material optionally comprises material not found with the material in its natural environment.

As used herein, “nucleic acid” or “oligonucleotide” includes reference to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides. In specific embodiments, the “nucleic acid” or “oligonucleotide” can be substituted by chemical substances that can form sequence specific interactions similar as for the natural phosphodiester “nucleic acid”. Known and preferred analogues include polymers of nucleotides with phosphorothioate or methylphosphonate liaisons, or peptid nucleic acids. Unless otherwise indicated, a particular nucleic acid sequence includes the complementary sequence thereof. Typical oligonucleotides are single-stranded nucleic acids of between 5 and 200 bases in length, more preferably of between 5 and 100, even more preferably of between about 10 and 50 bases. Examples of such oligonucleotides are single stranded DNA molecules of between 20 and 40 bases in length.

In the invention, a “probe” is a oligonucleotide that can be recognized by a particular target. In particular, and in preferred embodiments, the “probe” is immobilized on a surface. Depending on context, the term “probe” refers both to individual oligonucleotide molecules and to the collection of same-sequence oligonucleotide molecules surface-immobilized at a discrete location.

The term “target” refers to a nucleic acid molecule that has an affinity for a given probe. A target may be a naturally-occurring or a man-made nucleic acid molecule. It can be employed in their unaltered state or as aggregates with other species. Targets may be attached, covalently or noncovalently, to a binding member, either directly or via a specific binding substance. Targets may also be modified. In preferred embodiments, they harbor a fluorescent or radioactive moiety, or groups or isotopes that can be identified by mass spectrometry.

A “feature” according to the invention is defined as an area of a substrate having a collection of same-sequence, surface-immobilized oligonucleotide molecules. One feature is different than another feature if the probes of the different features have different nucleotide sequences.

The term “oligonucleotide array” refers to a substrate having a two-dimensional surface having at least two different features. Oligonucleotide arrays preferably are ordered so that the localization of each feature on the surface is spotted. In preferred embodiments, an array can have a density of at least five hundred, at least one thousand, at least 10 thousand, at least 100 thousand features per square cm. The substrate can be, merely by way of example, glass, silicon, quartz, polymer, plastic or metal and can have the thickness of a glass microscope slide or a glass cover slip. Substrates that are transparent to light are useful when the method of performing an assay on the chip involves optical detection. As used herein, the term also refers to a probe array and the substrate to which it is attached that form part of a wafer. The substrate can also be a membrane made of polyester or nylon. In this embodiment, the density of features per square cm is comprised between a few units to a few dozens.

The term “distinguishable phenotype” has to be understood as a phenotype (i.e. a qualitative or quantitative measurable feature of an organism) that can allow the categorization of a given population. For exemple, a distinguishable phenotype encompasses the membership to a set of a given disease, or a peculiar feature or property (e.g. resistance or adverse effect when given a given drug).

The future sequence of the human will be finished in the next couple of years. It will uncover the complete sequence of the 3 billion bases and the relative position of the 100 000 genes that constitute the genome. The enormous information revealed by this project opens unlimited possibilities for the elucidation of gene function and interaction of different genes. It will also allow the implementation of pharmacogenomics and pharmacogenetics.

Pharmacogenetics and pharmacogenomics aim at determining the genetic determinants linked to different phenotypes, in particular diseases. Most of the disease are multigenic diseases, and the identification of the genes involved therein should allow for the discovery of new targets and the development of new drugs. Pharmacogenomics also encompasses the use of specific medications according to the genotype of the patient. This should lead to a dramatic improvement of the efficiency of the drugs.

Many physiological diseases are targeted by this novel pharmaceutical approach. One can name the autoimmune and inflammatory diseases, for example Addison's Disease, Alopecia Areata, Ankylosing Spondylitis, Behcet's Disease, Chronic Fatigue Syndrome, Crohn's Disease and Ulcerative Colitis, Inflammatory Bowel Disease, Diabetes, Fibromyalgia, Goodpasture Syndrome, Lupus, Meniere's, Multiple Sclerosis, Myasthenia Gravis, Pelvic Inflammatory Disease, Pemphigus Vulgaris, Primary Biliary Cirrhosis, Psoriasis, Rheumatic Fever, Sarcoidosis, Scleroderma, Vasculitis, Vitiligo, Wegener's Granulomatosis.

Cancers are also believed to be multigenic diseases. Some oncogenes (for exemple ras, c-myc) and tumor suppressor genes (for exemple p53) have previously been identified, as well as some genetic markers for predisposition (for example the genes BRCA1 and BRCA2 for breast cancer). The identification of new genes involved in other kind of cancers should allow for a better information of the patient and the prevention of the development of the disease, an improved life expectancy as already observed with breast cancer (Schrag et al., JAMA, 2000; 283:617-24).

A necessary step for achieving these goals is therefore the characterization of the genetic determinants specific of a given genotype in a population of patients. The determination of variability at the genome level can be achieved by determining different markers and then refining the analysis to identify the genes of interest.

The major goal of genetics is indeed to link a phenotype (i.e. a qualitative or quantitative measurable feature of an organism) to a gene or a number of genes. Historically there are two genetics approaches that are applied to identify genetic loci responsible for a phenotype: familial linkage studies and association studies. Whatever the approach is, genetic studies are based on polymorphisms, i.e. base differences m the DNA sequence between two individuals at the same genetic locus.

Currently two kinds of markers are used for genotyping: microsatellites and single nucleotide polymorphisms (SNP). Microsatellites are highly polymorphic markers where different alleles are made up of different numbers of repetitive sequence elements between conserved flanking regions. On average, a microsatellite is found every 100 000 bases. A complete map of microsatellites markers covering the human genome was presented by the Centre d'Etude du Polymorphisme Humain (Dib et al., Nature 1996; 380:152-4). Microsatellites are genotyped by sizing PCR products generated over the repeat regions on gels. The most widely used systems are based on the use of fluorescently labeled DNA and their detection in fluorescence sequencers.

Fewer SNP are in the public domain, and a SNP map is currently being established by the SNP consortium which regroups pharmaceutical and electronics companies (Roberts, US News World Rep, 1999; 127:76-7).

Different analysis technologies have been developed for the genotyping of these markers, for example gel based electrophoresis, DNA hybridization, identification and characterization through mass spectrometry. The drawback of all these approaches is that they necessitate the amplification of many hundred of thoushands of specific sequences, which makes these technologies both labor intensive and expensive.

Linkage analysis has been the method of choice to identify genes implicated in many diseases both monogenic and multigenic, but where only one gene is implicated for each patient. In order to be reasonably powerful in the statical analysis the studied polymorphisms have to fulfill several criteria:

high heterozygosity i.e. many alleles exist for a given locus (this increases the informativity);

genome wide representation;

detectable with standard laboratory methods.

A type of polymorphisms fulfilling most of these criteria are microsatellite markers. As already described, these are repetitive sequence elements of two, three or four bases. The number of repetitions is variable for a given locus, resulting in a high number of possible alleles, i.e. high heterozygosity (70-90%). Microsatellite markers are still the genetic markers of choice for linkage analysis, and genotyping of these markers is performed by amplifying the alleles by PCR and size separation in a gel matrix (slab gel or capillary). For the study of complex human diseases usually 400-600 microsatellite markers are used that are distributed in regular distances over the whole genome (about 10-15 megabases).

Linkage studies follow alleles in families. However, each family might have a different allele of a genetic locus linked to the phenotype of interest. Association studies in contrast follow the evolution of a given allele in a population. The underlying assumption is that at a given time in evolutionaary history one polymorphism became fixed to a phenotype because:

a) it is itself responsible for a change in phenotype or,

b) it is physically very close to such an event and is therefore rarely separated from the causative sequence element by recombination (one says that the polymorphisms is in linkage disequilibrium with the causative event).

As association studies postulate the existence of one given allele for a trait of interest, it is therefore desirable that the markers for association studies are simple. Accordingly, the markers of choice are SNP, which show a simple base exchange at a given locus, and are therefore bi-, rarely tri-allelic. Association studies can be carried out either in population samples (cases vs controls) or family samples (parents and one offspring, where the transmitted alleles constitute the “cases” and the non-transmitted the “controls”).

In order to simplify the analysis and comparison of the genomes of two people bearing the same phenotype, and the potential identification of the genes linked to this phenotype, it can be interesting to reduce the complexity of the DNA samples to analyze. Such a method, called genomic mismatch scanning (GMS) was described by Nelson et al. (Nat Genet. 1993; 4:11-8). It allows the identification of all loci that are identical between two genomic DNA. This method will lead to a discrimination of the DNA samples, as only identical loci between two individuals will be present in solution after the GMS method is performed. The method of the invention will therefore be fully appreciated as it will allow the identification of said DNA samples, rather than their discrimination.

Other methods also lead to the reduction of the DNA complexity, for example degenerate oligonucleotide primer PCR, ALU-PCR or amplified restriction fragment length polymorphism (AFLP). Indeed, these methods are often used on genomic DNA to increase the amount of sample that would be needed for latter studies. The drawback of these methods is that certain parts of genomic DNA are not amplified by these techniques. This explains why one can consider that these methods reduce the complexity of genomic DNA. The method according to the present invention can be used to identify the regions of genomic DNA that have been amplified, and therefore the representation of said DNA compared to the whole genome.

Even with these methods, the analysis and comparison of the DNA samples remain labor intensive, as they necessitate a large number of PCR reactions, and gel analysis.

The invention provides a method which leads to the identification of specific DNA sequences from a mixture of DNA fragments, which allows to perform association and linkage studies. This method is simple, cheap and quick to perform.

The invention is drawn to a method for the identification of the presence of a genetic marker in a DNA sample comprising the following steps:

a) selection of sequences specific of said genetic marker;

b) fixation of oligonucleotides comprising said specific sequences or the complementary sequences on a solid support;

c) addition of a mixture of DNA fragments representing the said DNA sample to the solid support in a way that hybridization is possible;

d) detection of the presence of the genetic marker in the DNA sample by the presence of a signal corresponding to the hybridization of a fragment of the DNA sample to the specific oligonucleotide.

To perform the method of the invention, the sequences specific of the genetic marker are the flanking regions of said genetic markers. Indeed, even though the genetic marker is highly polymorphous in a population, its flanking regions are conserved between two individuals. This ensures that the study of the polymorphism of the genetic marker will not be hampered by poor hybridization.

The genetic marker which is looked for in the method described in the invention is preferably a SNP or a microsatellite, the latter being the most preferred case.

It has to be understood that the method of the invention is preferably to be used in genotypage studies, and that the presence or absence of the genetic marker of interest will be investigated in many individuals. Also, it is preferred if the genetic markers that are sought are linked to a distinguishable phentoype.

It has also to be understood that the method of the invention is not primarily intended to discriminate between multiple genetic markers, but rather to allow for the determination of the presence or the absence of said marker in a DNA sample, preferably a genomic DNA sample, the complexity of which has been reduced. In this regard, this invention is particularly directed at characterizing the content of (e.g., determining the presence or absence of a genetic marker in) a nucleic acid sample after said sample has undergone a selection process in which the complexity of said sample is reduced.

Nevertheless, and as could be described later, some improvement can be made to the current invention, that will further permit the identification of the genetic marker, the presence of which has been detected.

The current invention is also drawn to a method for the identification of gene(s) and/or mutation(s) associated with a distinguishable phenotype comprising the steps of:

a) identifying genetic markers associated with said phenotype, by applying the method described above to DNA samples from individuals exhibiting said phenotype;

b) comparing the regions identified in step a) with the corresponding regions in individuals that do not exhibit said phenotype;

c) identifying the gene(s) and/or mutation(s) associated with said phenotype.

The first step will allow to determine the shared genetic markers between two individuals exhibiting a given phenotype (population A). It can therefore be postulated that the genetic marker linked to said phenotype can be isolated by this step. In order to refine the analysis, the step b) compares the genetic markers isolated in step a) with the markers harbored by individuals that do not exhibit the phenotype (population B). Therefore, any genetic marker shared between population A and population B is not linked to the phenotype. The use of this method with a sufficient number of individuals allows the restriction to a small number of genetic markers and the identification of the gene(s) and/or mutation(s) linked to the phenotype of interest.

It is as well very preferable to have reduced the complexity of the DNA genomes to compare. It might be best to perform the method of GMS between two individuals, as this method reduces the DNA samples to be analyzed to the DNA fragments that are identical between the two individuals. But the other methods of reduction of complexity described above could also be used favorably.

This method is best performed on individuals that are related (i.e. from the same family, in a large meaning, parents, cousins, uncles, aunts . . . ). In fact, this is preferable, as related individuals share a certain percentage of DNA (on average 50% between brothers and sisters, 16% between cousins). Therefore, it is more likely that they will have identical genetic markers if they share the same phenotype, and that these markers will be missing from the related individuals that do not exhibit the phenotype. By comparison of the missing hybridization spots, it will allow a very quick determination of the genetic markers linked to the phenotype.

In a particular embodiment, this invention relates to a method of identifying genes and/or mutations associated with a phenotype or trait, the method comprising:

(a) preparing a composition enriched for identical nucleic acid fragments from nucleic acid samples from individuals exhibiting said phenotype,

(b) characterizing said composition by contacting the same with a nucleic acid array of oligonucleotides specific for flanking regions of selected genetic markers.

The present invention also includes methods of identifying genes related to a phenotype, the methods comprising:

(a) isolating nucleic acid fragments that are identical between two individuals exhibiting said phenotype, and

(b) identifying genes contained in said nucleic acid fragments by contacting said fragments with a nucleic acid array comprising, on a support, nucleic acid sequences specific for regions flanking genetic markers.

Step (a) is preferably performed by a genomic mismatch scanning (“GMS”) approach, as described previously or by comparative genomic hybridisation (“CGH”). Alternatively, step (a) can be accomplished using the method described in WO00/53802. Most preferably, step (a) comprises treating the sample to produce IBD fragments. The method is particularly suited to identify genes or mutations from genomic DNA from said individuals. In a particular embodiment, the genomic DNA or fragments may be amplified.

A preferred use of the above methods is to identify genes or mutations related to a pathological condition, particularly a cardiovascular disease, lipid-metabolism disorder or central nervous system disorder.

Furthermore, in a particular embodiment, the method further comprises the step of comparing the genes identified in (b) with the sequence of corresponding genes from individuals that do not exhibit the phenotype.

The present invention also relates to kits for implementing a method as described above, comprising a nucleic acid array and reagents to isolate identical nucleic acid fragments from two samples.

The invention also relates to the use of a gene or mutation identified by a method as described above, for diagnotic, therapeutic or screening purposes. The genes or mutations can be used to design probes or primers suitable to detect the presence of said gene or mutation in any sample. Identification of said gene or mutation in a sample from a subject may indicate the presence of or predisposition to a pathology. The gene or mutation may allow one to design a gene therapy product incorporating the wild type version or any antisens product, to correct the deficiency associated with said gene or mutation. The gene or mutation also allows the implementation of screening methods to identify compounds that regulate the activity or expression of said gene.

In a preferred embodiment of the above methods according to this invention, the oligonucleotides comprising the sequences specific of the genetic marker are further used for the amplification of said genetic marker. The characterization of the amplified product can be carried out with the usual methods known by the person skilled in the art (in particular electrophoresis, chromatography, sequencing, or mass spectrometry).

In order to improve the hybridization properties, it might be useful to modify the oligonucleotides, in particular to substitute them by chemical substances that can form sequence specific interactions, as previously described.

One understands that the methods described in the current invention are best performed by using DNA arrays. These arrays of oligonucleotides comprising sequences specific of genetic markers, in particular the flanking sequences of said genetic marker, are also part of the invention. Most preferably, the genetic marker is a microsatellite marker.

It is highly preferable to prepare an array comprising all the flanking sequences specific of the genetic markers the presence of which the investigator wants to determine. In particular, an array comprising oligonucleotides comprising the flanking sequences (or complementary sequences) of all the microsatellite markers will be of choice for performing the methods of the invention. The array may comprise between 100 and 200 000 oligonucleotides specific for said sequences. The array may comprise oligonucleotides specific for different types of genetic markers, e.g., SNPs and microsatellites.

The map of the microsatellite markers and their sequences can easily be determined by the person skilled in the art (Dib et al., Nature 1996; 380:152-4), which can determine the flanking sequences specific of each microsatellite that are suitable for use on a DNA array, in the methods according to the invention. It is indeed important for the melting point of the oligonucleotides to be in the same range for each oligonucleotide, in order to improve the quality of hybridization. Preferred flanking regions of the genetic markers correspond to regions located within 500 bp at the most on each side of the genetic marker.

The construction of the oligonucleotide array can be carried out by using methods known by the one skilled in the art. In particular, the synthesis can be performed directly on the solid surface, in particular by a photochemical (U.S. Pat. No. 5,424,186) or an ink-jet technique. Alternatively, the oligonucleotides can be synthesized ex situ and further bound to the solid surface. In this case, it might be useful for the oligonucleotide to carry a chemical modification that allows the binding to the solid surface. The addressing of the oligonucleotides on the surface can be performed mechanically, electronically or by ink-jet.

The hybridization conditions will depend on the DNA sample to be analyzed, but can be easily optimized by the person skilled in the art. The conditions can be optimized by modifying the salinity, pH and temperature of hybridization. They can also be electronically assisted (U.S. Pat. No. 6,017,696), in order to improve the specificity.

The detection of the hybridization spots can be performed by radioisotopic or fluorescent labeling, field effect measurement, opto-electrochemical process, piezzo-electrical process, or ellipsometry, optical fibers measurement, mass spectrometry.

An alternative to oligonucleotide arrays can be the use of silicon microbeads on which the oligonucleotides of the invention are bound. In this case, it is advantageous to perform the detection of hybridization events by telemetry. It is preferable when each bead harbors a specific code, the reading of said code allowing the identification of the hybridization events.

Prior to hybridization, it might be advantageous to label the DNA fragments with fluorescent dyes or radioisotopes in order to facilitate the detection with these techniques. Alternatively, it can be interesting to label these fragments, prior to hybridization, with groups or isotopes that can be identified by mass spectrometry, in the case the detection is done by this method. The person skilled in the art knows the moieties and/or groups to use for such a purpose. It is highly desirable to use base specific labels.

In another embodiment, the DNA fragments are labeled subsequently to hybridization, by the use of a proofreading DNA polymerase and labeled di-desoxy nucleotides (ddNTP), that leads to primer extension of the oligonucleotide. The person skilled in the art knows that this extra step increases the specificity of the reaction (Pastinen et al. Genome Res., 1997, 7, 606). The primer extension reaction is performed on the immobilized oligonucleotide if a DNA template is hybridized to it with nucleotides labeled with fluorescent dyes, radioactive isotopes, or groups or isotopes that can be identified by mass spectrometry. The use of different fluorescent dyes or different masses of groups added to the ddNTP's in the primer extension reaction further increase the specificity and allow the unambiguous identification of a specific fragment hybridization from background hybridization, and therefore to the presence of the genetic marker.

In the case the genetic marker the presence of which has been determined is a SNP, this extra step of primer extension can also allow the identification of said SNP, as the use of ddNTPs labeled with different markers (preferably different fluorescent dyes) can lead to the unambiguous determination of said SNP base.

The methods according to the invention are useful to determine the gene(s) and/or mutation(s) responsible for a distinguishable phenotype. For example, they can be carried out on human beings, in order to quickly identify the genetic marker(s) responsible for a given disease, or a susceptibility to a disease. They can also be carried out in the agricultural field, on animals or plants. The investigator can, with these methods, determine the genotype of animals or plants presenting an interest for the farmer and/or the industrial, and improve the quality of the products. For example, it could be interesting to determine the gene(s) responsible for a high casein concentration in dairy cattle.

The method can also be used on smaller organisms, like bacteria, viruses or parasites, for example in order to quickly identify the mutation(s) in the genes that are linked to drug resistance. The person skilled in the art knows how to choose the oligonucleotides to perform this method in this case.

The methods described in the current invention offer obvious advantages over the classical linkage and association methods.

The methods allow unambiguous detection of IBD fragments between individuals, and is not dependent on allele frequencies or marker heterozygosity;

These methods are not limited to the use of polymorphic markers, and can be performed with any sequence, as long as some sequence and ampping information is available:

The information given by these methods is based on the presence or absence of a hybridization signal. This is an important advantage compared to the methods of the technique that necessitates allele discrimination.

After determination of a region of interest, for example by using the microsatellites, the same methods can be applied to reduce the size of the region and identify the fragments of interest. This scaling to any density of the genome is very valuable.

Due to these advantages, it is necessary to screen less individuals to perform the methods described in the current invention, and obtain usable results. This is particulary true when related individuals are tested, and when the GMS method is first performed on their DNA.

The following examples illustrate some preferred embodiments of the invention, but shall not be considered as restricting the scope of the invention.

### DESCRIPTION OF THE FIGURE

FIG. 1 represents the microsatellite D1S2729 (underlined) and its flanking regions (SEQ ID N^{o} 1). Two oligonucleotides that can be chosen in the flanking regions in order to perform the method according to the invention are represented by arrows (1.A.). FIG. 1.B represents the chemical modifications that can be added to the oligonucleotides in order to fix them on a solid support. The presence of microsatellite D1S2729 in the DNA sample after GMS reduction will lead to its hybridization to the oligonucleotides and to the presence of a fluorescent signal that can be detected.

### EXAMPLES

### Example 1

### Reduction of DNA Complexity by GMS

Genomic DNA from subjects in a collection of families where at least two related individuals show the same disease phenotype, is extracted by standard methods e.g. phenol-chloroform extraction. The DNA's are separately cut with a restriction enzyme (e.g. PstI) to create restriction fragments with an average size around 4 kilobases. To one of each of the restriction mixes from a pair of individuals a solution containing dam methylase is added and the DNA is methylated at adenin bases. The methylated products from one individual are then mixed with the non-methylated product of the second subject from the same family. The products are then heat denatured and allowed to re-anneal using stringent hybridisation conditions (Casna et al. (1986) Nucleic Acids Res. 14:7285-7303). This results in the formation of heteroduplexes from the DNA's from different sources (individuals) which are hemimethylated (hybridisation of one methylated strand with one non-methylated. In addition homoduplexes are formed by renaturation between the strands of each individulal with itself. These homoduplexes are either completely methylated or completely non-methylated.

Using methylation sensitive enzymes like MboI (only cuts methylated double stranded DNA) and DpnI (only cuts unmethylated double stranded DNA) the homohybrids are digested. To this mixture a solution containing exo III (or an equivalent 3′ recessed or blunt-end specific exonuclease) exonuclease is added. The exonuclease digests the blunt ended digested homoduplex fragments but not the heteroduplexes with their 3′ overhang, creating big single stranded gaps in the homoduplex fragments. These can be eliminated from the reaction mix through binding to a single strand specific matrix (e.g. BND cellulose beads).

The remaining heteroduplexes comprise a pool of 100% identical fragments and fragments with base pair mismatches (non-IBD fragments). A solution containing the mismatch repair enzymes mutSHL is added to the mix resulting in the nicking of mismatched heteoduplexes at a specific recognition site (GATC). These nicks are further digested by adding exo III (or an equivalent 3′ recessed or blunt-end specific exonuclease) exonuclease to the reaction mix, creating big single stranded gaps in the homoduplex fragments. These can be eliminated from the reaction mix through binding to a single strand specific matrix (e.g. BND cellulose beads).

The remaining fragments in the reaction mix constitute a pool of 100% identical DNA hybrids formed between the DNA's of different individuals comprising the loci responsible for the disease phenotype.

### Example 2

### Manufacture of an Oligonucleotide Array

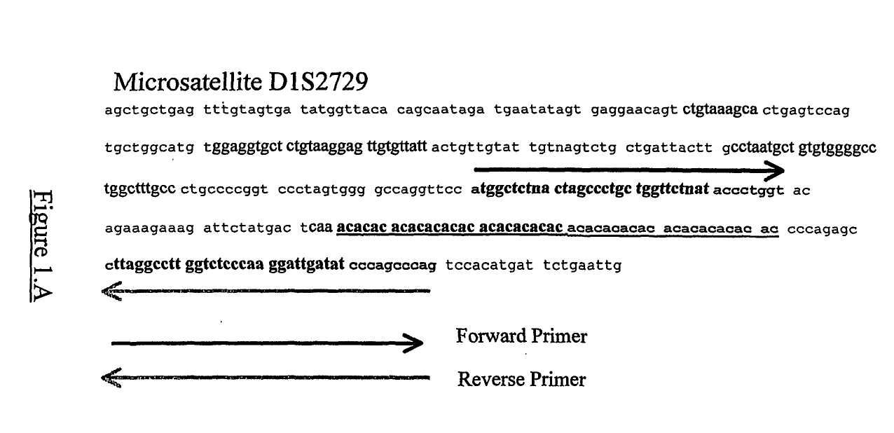

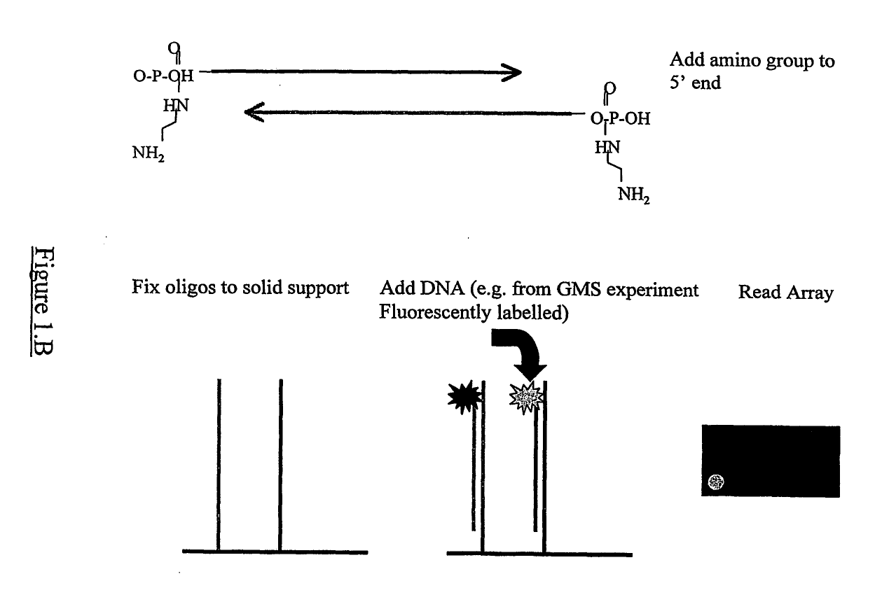

From the human genetic map which links over 5000 microsatellite markers forward and reverse sequences flanking the repeat units are selected The selection is carried out from sequence information available through public data bases especially the GENETHON database (FIG. 1). Critera for selection are the uniqueness of the sequences in respect to each other, common primer selection criteria for hybridization (no self-complementarity, similar Tm etc.) and sequence stability (no known polymorphic sites in the oligonucleotide sequence.

The corresponding sequences are then synthesized in the form of oligonucleotides that are typically between 25 and 35 bases long and are activated by the addition of an amino group to their 5′ end (e.g. by addition and are synthesized by standard procedures by a manufacturer providing salt free, high quality oligonucleotides (e.g. MWG, Germany)).

These oligonucleotides are then applied to an amino-silane covered glass slide using an appropriate automated arrayer (e.g. GMS 417 Arrayer, Genetic Microsystems), through a specific reaction (see e.g. Urdea et al. Nucleic Acids Res. 11 (1988)). An aminoester bridge is formed between the oligonucleotide and the aminosilane and the oligonucleotide thus bound to the glass slide.

This array constitutes a representative selection of the whole human genome with an average resolution of <1 cM (sex averaged, about one marker every 1 megabase).

### Example 3

### Hybridization Protocol

The remaining hybrid fragments are hybridized against the microsatellite array in a hybridization chamber in a hybridization buffer (e.g. 6×SSC, 5× Denhardt's solution), at temperatures between 45-62° C. After hybridization several washes with icreasing stringency (3-0.1×SSC, 0.05% Tween 20 at 37-45° C.) are carried out to wash out non-specific hybridizations. The person skilled in the art can optimize the hybridization conditions, in particular with the teachings of Sambrook et al. (1989; Molecular cloning: a laboratory manual. 2^{nd} Ed. Cold Spring Harbor Lab., Cold Spring Harbor, N.Y.).

### Example 4

### Primer Extension Protocol

To increase the specificity a solution of fluorescently labelled didesoxynucleotides is added where each of the four ddNTP's carries a different fluorophore. Through a polymerase the subsequent base following the last base on the oligonucleotide that is fixed to the chip is added. The DNA polymerase used (T7, Taq, Klenow fragment . . . ) and the polymerization conditions will be chosen by the person skilled in the art depending on the DNA fragments to extend and according to the teaching of Sambrook.

### Example 5

### Detection Protocol

The result is the identification of fragments still present after the GMS procedure by both position and fluorescent signal (colour). Statistical analysis of the signals from a sufficiently large number of families identifies the loci common to affected individuals within a narrow interval of a few cMorgan.

1. A method for the identification of the presence of a genetic marker in a DNA sample comprising the following steps:
a) selection of sequences specific of said genetic marker;
b) fixation of oligonucleotides comprising said specific sequences or the complementary sequences on a solid support;
c) addition of a mixture of DNA fragments representing the said DNA sample to the solid support in a way that hybridization is possible;
d) detection of the presence of the genetic marker in the DNA sample by the presence of a signal corresponding to the hybridization of a fragment of the DNA sample to the specific oligonucleotide,
wherein said specific sequences are flanking sequences of said genetic marker and said DNA sample has been reduced in complexity.

2. The method of claim 1 wherein the genetic marker is a microsatellite marker.

3. The method of claim 1 wherein the genetic marker is a single nucleotide polymorphism (SNP).

4. The method of any of claims 1 to 3 wherein said oligonucleotides are further used for the amplification of said genetic marker.

5. The method of any of claims 1 to 3 wherein the hybridization step is followed by a primer-extension step.

6. The method of any of claims 1 to 5 wherein said oligonucleotides are substituted by chemical substances that can form sequence specific interactions.

7. The method of any of claims 1 to 6 wherein the selected sequences are bound to the solid phase in an ordered fashion.

8. The method of claim 7 wherein the solid phase is a two-dimensional surface.

9. The method of claim 7 wherein the solid surface is an individually coded bead.

10. The method of any of claims 1 to 9 wherein said DNA sample has been reduced in complexity by isolation of identical fragments from two individuals.

11. The method of claim 10 wherein the DNA sample has been reduced in complexity by the method of Genomic Mismatch Scanning.

12. The method of any of claims 1 to 11, wherein the detection is performed by radioisotopic or fluorescent labeling, field effect measurement, opto-electrochemical process, piezzo-electrical process, or ellipsometry, telemetry, optical fibers measurement, mass spectrometry.

13. The method of any of claims 1 to 12 wherein the genetic marker is associated with a distinguishable phenotype.

14. A method for the identification of gene(s) and/or mutation(s) associated with a distinguishable phenotype comprising the steps of:
a) identifying of genetic markers associated with said phenotype, by applying the method of any of claims 1 to 13 to DNA samples from individuals exhibiting said phenotype;
b) comparing the regions identified in step a) with the corresponding regions in individuals that do not exhibit said phenotype;
c) identifying the gene(s) and/or mutation(s) associated with said phenotype.

15. The method of claim 14, wherein the individuals exhibiting and the individuals that do not exhibit said phenotype are related.

16. A method of identifying genes related to a phenotype, the method comprising:
(a) isolating nucleic acid fragments that are identical between two individuals exhibiting said phenotype, and
(b) identifying genes contained in said nucleic acid fragments by contacting said fragments with a nucleic acid array comprising, on a support, nucleic acid sequences specific for regions flanking genetic markers.

17. The method of claim 16, wherein said phenotype is a pathological condition, particularly a cardiovascular disease, lipid-metabolism disorder or central nervous system disorder.

18. The method of claim 16 or 17, wherein step a) comprises isolating identical nucleic acid fragments from genomic DNA from said individuals.

19. The method of claim 18, wherein the genomic DNA or fragments are amplified.

20. The method of claim 18, wherein said isolation is obtained by GMS or CGH.

21. The method of any one of claims 16-20, further comprising the step of comparing the genes identified in (b) with the sequence of corresponding genes from individuals that do not exhibit the phenotype.

22. The use of a gene or mutation identified by a method of any one of the preceding claims, for diagnotic, therapeutic or screening purposes.

23. A kit for implementing a method of any one of claims 1 to 21, comprising (i) a nucleic acid array comprising, on a support, nucleic acid sequences specific for regions flanking genetic markers and (ii) reagents to isolate identical nucleic acid fragments from two samples.

24. A method for the identification of the presence of a genetic marker in a DNA sample comprising the following steps:
selection of sequences specific of said genetic marker;
fixation of oligonucleotides comprising said specific sequences or the complementary sequences on a solid support;
addition of a mixture of DNA fragments representing the said DNA samplt to the solid support in a way that hybridization is possible;
detection of the presence of the genetic marker in the DNA sample by the presence of a signal corresponding to the hybridization of a fragment of the DNA sample to the specific oligonucleotide,
wherein said specific sequences are flanking sequences of said genetic marker and said DNA sample has been reduced in complexity.

25. The method of claim 24, wherein the genetic marker is a microsatellite marker.

26. The method of claim 24, wherein the genetic marker is a single nucleotide polymorphism (SNP).

27. The method of claim 24, wherein said oligonucleotides are further used for the amplification of said genetic marker.

28. The method of claim 24, wherein the hybridization step is followed by a primer-extension step.

29. The method of claim 24, wherein said oligonucleotides are substituted by chemical substances that can form sequence specific interactions.

30. The method of claim 24, wherein the selected sequences are bound to the solid phase in an ordered fashion.

31. The method of claim 30, wherein the solid phase is a two-dimensional surface.

32. The method of claim 30, wherein the solid surface is an individually coded bead.

33. The method of claim 24, wherein said DNA sample has been reduced in complexity by isolation of identical fragments from two individuals.

34. The method of claim 33, wherein the DNA sample has been reduced in complexity by the method of Genomic Mismatch Scanning.

35. The method of claim 24, wherein the detection is performed by radioisotopic or fluorescent labeling, field effect measurement, opto-electrochemical process, piezzo-electrical process, or ellipsometry, telemetry, optical fibers measurement, mass spectrometry.

36. The method of claim 24, wherein the genetic marker is associated with a distinguishable phenotype.

37. A method for the identification of gene(s) and/or mutation(s) associated with a distinguishable phenotype comprising the steps of:
identifying of genetic markers associated with said phenotype, by applying the method of claim 24 to DNA samples from individuals exhibiting said phenotype;
comparing the regions identified in step a) with the corresponding regions in individuals that do not exhibit said phenotype;
identifying the gene(s) and/or mutation(s) associated with said phenotype.

38. The method of claim 37, wherein the individuals exhibiting and the individuals that do not exhibit said phenotype are related.

39. A method of identifying genes related to a phenotype, the method comprising:
isolating nucleic acid fragments that are identical between two individuals exhibiting said phenotype, and
identifying genes contained in said nucleic acid fragments by contacting said fragments with a nucleic acid array comprising, on a support, nucleic acid sequences specific for regions flanking genetic markers.

40. The method of claim 39, wherein said phenotype is a pathological condition, particularly a cardiovascular disease, lipid-metabolism disorder or central nervous system disorder.

41. The method of claim 39 or 40, wherein step a) comprises isolating identical nucleic acid fragments from genomic DNA from said individuals.

42. The method of claim 41, wherein the genomic DNA or fragments are amplified.

43. The method of claim 41, wherein said isolation is obtained by GMS or CGH.

44. The method of claim 39, further comprising the step of comparing the genes identified in (b) with the sequence of corresponding genes from individuals that do not exhibit the phenotype.

45. A kit for implementing a method of any claim 24, 37 or 39, comprising (i) a nucleic acid array comprising, on a support, nucleic acid sequences specific for regions flanking genetic markers and (ii) reagents to isolate identical nucleic acid fragments from two samples.
